(19) United States
(12) Patent Application Publication
(10) Pub. No.: US 2003/0108911 A1
(43) Pub. Date: Jun. 12, 2003

(54) Arrangement and method for multiple-fluorescence measurement

(75) Inventors: Ingo Klimant, Mintraching (DE); Jens Kurner, Regensburg (DE)

Correspondence Address: HENRY M FEIEREISEN, 350 FIFTH AVENUE, SUITE 3220, NEW YORK, NY, 10118, US

(73) Assignee: CHROMEON GmbH

(21) Appl. No.: 10/209,417

(22) Filed: Jul. 31, 2002

(30) Foreign Application Priority Data
Aug. 1, 2001 (DE) 101 37 530.1

(51) Int. Cl. C12Q 1/68; G01N 33/53; C12M 1/34; C07F 15/00

(52) U.S. Cl. 435/6; 435/7.1; 546/2; 435/287.2

(57) ABSTRACT

In nanoparticles, a phosphorescent donor dyestuff and several fluorescent acceptor dyestuffs are immobilized together. These nanoparticles serve as multiplex marker for a number of analytes, which can be determined according to absorption spectra of the acceptor dyestuffs as well as according to the luminescence-decay period of the respective dyestuffs.

### DESCRIPTION

The invention relates to an arrangement and a method for multiple-fluorescence measurement by means of a multitude of fluorescence markers commonly immobilized, in particular in micro- and nanoparticles. The dyestuffs have overlapping absorption spectra, so that with excitation of a dyestuff an energy transfer to the adjacent dyestuff takes place. The measurable light yield is thereby multiplied which renders these particles suitable for a highly sensitive detection of substances, in particular biomolecules to which they are bound, for instance for detection of RNA or DNA, in the flow-through zytometry, microscopic analyses techniques such as light or fluorescence microscopy or, also confocal 3D-microscopy for diagnostics, in analyses, medicine and immunoassays.

The dyestuffs are selected in such a manner, that they realize a possibly large Stokes-shift at a high light yield, in order to separate excitation and signal of the dyestuffs without extensive light losses.

Further important properties of the dyestuffs are a long-term photostability. The luminescence properties should not be influenced by the sample. Reactive groups must be available, in order to selectively couple to the molecule to be determined. The dyestuffs should be water soluble and non-toxic.

Although a number of different fluorescence dyestuffs are known to be markers, it has been shown that only few of these dyestuffs fulfill all the afore-recited criteria.

Problematic in particular is an insufficient brightness of the measuring signals, especially with such samples that have a high background fluorescence.

Furthermore, there is a great demand for various dyestuffs with clearly varying features (multiplex-dyestuffs) for differentiating a great number of differently labeled biological samples from each other, as for example DNA-fragments or proteins.

In order to elevate the brightness of luminescence assays and to eliminate the inherent background fluorescence in the sample, the fluorescence dyestuffs can be incorporated into the afore-described polymer matrices, for instance micro-or nanoparticles, thereby not only raising the quantum yield, but at the same time protecting the dyestuffs from the unwanted influences of the matrix, in particular quenching. Otherwise, the incorporation of a multitude of different dyestuff molecules into a single particle makes possible a distinct elevation of the signal intensity in a luminescence assay.

To improve the brightness and to eliminate the background fluorescence of the sample, furthermore, long wave-emitting luminescence dyestuffs can be utilized. With these, the selective detection of luminescence signals in natural samples can be realized, such as for example body fluids, since only very few natural compounds emit red light.

A further possibility to improve the brightness and to eliminate the background fluorescence, is the use of phosphorescent dyestuffs. Since the inherent fluorescence in most samples is normally completely decayed after a few nanoseconds, with the use of phosphorescent dyestuffs, due to their extended decaying time, a time terminated measurement and thus a background fluorescent-free detection of fluorescent signals is realized.

Typically, the chelates of the rare earth metals (Eu3+, Tb3+) are often-used fluorescence dyestuffs.

The afore-described so-called multiplex-dye stuffs or multiplex-markers can be produced conventionally as follows:

1. Use of a series of dyestuffs having various spectral properties with respect to absorption and emission.

2. Use of microparticles, each with several incorporated dyestuffs having the same absorption—but different emission properties.

3. Use of microparticles with two incorporated dyestuffs, varying spectrally from each other, such as identification via radiometric measurement of two luminescence intensities; and

4. Use of a series of dyestuffs with varying decay-behavior, but having identical spectral properties.

All these conventional concepts are however subject to limitations: Only a limited number of different markers can be produced, maximally 6 to 10. Furthermore, the afore-described concepts 1, 2 and 4 require for each individual marker an individual fluorescence dyestuff, whereas concept 3 at least requires only two individual dyestuffs in order to provide a whole series of markers.

For the multianalyte-detection, which is rapidly gaining importance, especially in DNA- and immuno-analytics, a substantially larger number of clearly distinguishable dyestuff markers is necessary, in particular for sorting cells, for flow-through zytometry, for immuno-and DNA-chips and for the fluorescence microscopy.

From U.S. Pat. No. 5,326,692, it is known to immobilize a cascade of spectrally overlapping fluorescence dyestuffs in nanoparticles.

An object of the invention is thus, to provide an arrangement and a method for fluorometric measurement of a sample, with which a less problematic multiple measurement of a multitude of luminescence signal can be realized.

As a solution to this object, it is proposed to provide a luminescent donor dyestuff and several acceptor dyestuffs which are immobilized with the donor dyestuff and which luminesce through energy transfer from the donor dyestuff, wherein the donor dyestuff is provided as a phosphorescence dyestuff and the respective acceptor dyestuffs are effected as fluorescence dyestuffs.

In contrast to U.S. Pat. No. 5,326,692, the donor dyestuff used is not a fluorescence dyestuff but a phosphorescence dyestuff, whereas the acceptor dyestuffs can be selected from the commonly used fluorophores. The broad emission band of phosphorescence dyestuffs used a donors permit combining this donor with a number of different acceptor dyestuffs in order to obtain spectrally different properties.

Each donor/acceptor pairing reacts in a predetermined way on a specific analyte.

Despite use of several different acceptor dyestuffs, a single donor dyestuff suffices, preferably a highly luminescent Ru(II)-polypyridyl complex, which can be combined with these differing acceptor dyestuffs. Preferably, a donor dyestuff with a long luminescence decay time of, for example, 100 ns to 100 μs, in particular preferably a long luminescence decay time relative to the emission stimulated by the donor dyestuff, for example, ≧50 ns, preferably 50 ns to 10 μs.

The several different acceptor dyestuffs, that are immobilized together with the donor dyestuff, can vary in their emission spectra. In that way, a one-dimensional series of fluorescence markers with identical absorption behavior but spectrally clearly differentiated emission properties can be obtained. If additionally, the concentration of the acceptor dyestuff is varied, so that each acceptor dyestuff is separately immobilized in several different concentrations with the donor dyestuff, the temporal decay behavior of the donor dyestuff likewise changes and at the same time also the temporal decay behavior of the stimulated fluorescence of the respective acceptor dyestuff, is changed.

Thereby it is possible, apart from the spectral properties of the acceptor dyestuff, to utilize also the luminescence decay times of the arrangement, that is, the donor dyestuff and/or the respective acceptor dyestuff as a parameter for identifying the analyte. Thus a two dimensional field of luminescence markers is realized, wherein the first dimension is defined by the spectral emission properties of the acceptor and the second dimension-through the temporal decay behavior of the donor and/or the acceptor in dependence of the respective concentration.

The acceptor dyestuffs are preferably carbocyanine dyestuffs, of which a multitude of variants is commercially available. These carbocyanine dyestuffs do not exhibit any inherent absorption at the excitation wavelength of the Ruthenium complex of the donor, namely at 488 nm. Up to ten different acceptor dyestuffs can be immobilized at the same time with a common donor dyestuff.

Preferably, the donor dyestuff and the acceptor dyestuff are immobilized together with a plastic matrix, wherein the donor dyestuff can have a concentration from 1 to 15% by weight, preferably about 10% by weight, without significantly reducing the quantum yield. The lack in overlap of absorption and emission of the donor molecule prevents a self-cancellation. This leads to an extremely high brightness of the luminescence signals.

Preferably, the donor dyestuff and the acceptor dyestuffs are embedded into micro- or nanoparticles, preferably of a size of about ≦50 μm, consisting of polymerized monomers, e.g. acrylates, styrols, unsaturated chlorides, esters, acetates, amides, alcohols etc. especially such as polymethyl-methacrylate-particles or those made from polystyrol. The particles can also be coated for modifying their surface structure.

These micro-or nanoparticles can be produced by precipitation of a solution of polynitril in dimethylformamide (DMF), wherein at the same time the dyestuffs are embedded into the particles.

The invention relates further to a process for the simultaneous fluorometric measurement of several analytes, in particular with an arrangement as afore-described with the steps of:

excitation of the donor dyestuff and

measurement and evaluation of the spectrally different fluorescence responses of the acceptor dyestuffs, and

measurement and evaluation of luminescence decay times of the arrangement influenced by the fluorescence signals of the acceptor dyestuffs in interaction with the donor dyestuff.

The fluorescence responses, respectively the fluorescence decay periods can be correlated to the presence, respectively the concentration, of the analytes to be determined, in accordance with basically known methods.

Thus, a two-dimensional field of luminescence markers is obtained, which is defined through the spectral behavior of the acceptor dyestuffs and the temporal behavior of the arrangement.

The measurement and evaluation of the fluorescence responses of the acceptor dyestuffs or/and the luminescence decay times which are influenced by the fluorescence signals of the acceptor dyestuffs in interaction with the donor dyestuff, is carried out preferably time-resolved, in order to reduce the background signal. Particularly preferred a temporal measuring window is adjusted, so that the measurement starts only after substantial decay of the background signal, with a short decay period of for example <50 ns.

In accordance with the measurement arrangement of the invention, the following properties are obtained.

1. The fluorescence of the acceptor induced through energy transfer slowly decays, namely in the range of microseconds, and thereby carries the temporal decay properties of the phosphorescence. With the time-terminated methods of the phosphorescence detection, a background-free measuring is thus realized.

2. A 2D-field of fluorescence markers can be realized. With seven different dyestuffs (one donor, six acceptors) and ten individually distinguishable decay periods, 60 distinguishable markers can be realized.

3. The emissions of all markers can be excited with a blue argon ionlaser. Due to the especially efficient light absorption of the ruthenium complex as donor, also at a wavelength of 404 nm, blue laser diodes can also be utilized as light source.

4. The Stokes-shift of all markers is exceptionally large. When using the blue light diodes as light source, the Stokes-shift is between 190 nm and 360 nm. According to U.S. Pat. No. 5,326,692, this would be obtained only with an extremely long cascade of many dyestuffs, whereby a great loss in brightness would occur, since each cascade step causes an additional loss in signal. These large Stokes-shifts are due to the spectral properties of the donor.

5. The incorporation of the phosphorescent donor molecules into a polymer matrix with a small oxygen-permeability prevents a cancellation of the phosphorescence and improves the signal intensities.

6. Through use of polyacrylonitrile-copolymerizate as matrix, phosphorescent nanoparticles can be produced having reactive surfaces for the coupling of biomolecules. The loading density of the surfaces having reactive groups can be adjusted through the properties of the co-polymer.

7. Various particles can be utilized, for example also latex particles, that are subsequently dyed, wherein the incorporation of the dyestuff occurs during the emulsion-polymerization.

Following is a description of examples of embodiments of the invention.

1. Preparation of the Starting Solutions

For production of the dyestuff solutions A, B1 to B4 and C1a to C1e, C2a to C2e, C3a to C3e and C4a to C4e were produced by the batches as listed in Tables 1 to 6. The following abbreviations apply:

| | |
|---|---|
| RU | Ru(dph-phen)₃ (TMS)₂ as donor dyestuff |
| PAN-COOH | poly-(acrylonitrile-co-acrylic acid) (5% by weight acrylic |
| | acid) as matrix material |
| CY582 | 3,3′-diethyloxadicarbocyanine-iodide (99%) as acceptor |
| CY604 | 1,1′-diethyl-2,2′-carbocyaninechloride as acceptor |
| CY655 | 3,3′-diethylthiadicarbocyanine-iodide (98%) as acceptor |
| CY703 | 1,1′-diethyl-4,4′-carbocyanine-iodide (96%) as acceptor. |

The polyacrylonitrile matrix and the ruthenium- and carbocyanine dyestuffs are completely soluble in N,N-dimethylformamide (DMF) as solvent.

TABLE 1
Production of the Ruthenium Donor Solution A

| Solution | A |
|---|---|
| M (RU) [g/mol] | 1,404.80 |
| m (RU) [mg] | 7.024 |
| n (RU) [μmol] | 5.0 |
| m (PAN-COOH) [g] | 1.0 |
| V (DMF) [ml] | 100 |

TABLE 2
Production of the Carbocyanine-acceptor Solutions B1 to B4

| Solution | B1 | B2 | B3 | B4 |
|---|---|---|---|---|
| dye | CY582 | CY604 | CY655 | CY703 |
| M (dye) [g/mol] | 486.36 | 388.94 | 518.48 | 480.39 |
| m (dye) [mg] | 6.0 | 5.0 | 5.0 | 20.0 |
| n (dye) [μmol] | 12.3 | 12.9 | 9.6 | 41.6 |
| V (DMF) [mL] | 60 | 50 | 50 | 75 |

TABLE 3
Production of Energy Transfer Solutions C1a-C1e

| Solution | C1a | C1b | C1c | Cld | C1e |
|---|---|---|---|---|---|
| V (A) [mL] | 5.0 | 5.0 | 5.0 | 5.0 | 5.0 |
| V (B1) [mL] | 0 | 0.5 | 1.0 | 2.5 | 5.0 |
| V (DMF) [mL] | 5.0 | 4.5 | 4.0 | 2.5 | 0 |

TABLE 4
Production of Energy Transfer Solutions C2a-C2e

| Solution | C2a | C2b | C2c | C2d | C2e |
|---|---|---|---|---|---|
| V (A) [mL] | 5.0 | 5.0 | 5.0 | 5.0 | 5.0 |
| V (B2) [mL] | 0 | 0.5 | 1.0 | 2.5 | 5.0 |
| V (DMF) [mL] | 5.0 | 4.5 | 4.0 | 2.5 | 0 |

TABLE 5
Production of the Energy Transfer Solutions C3a-C3e

| Solution | C3a | C3b | C3c | C3d | C3e |
|---|---|---|---|---|---|
| V (A) [mL] | 5.0 | 5.0 | 5.0 | 5.0 | 5.0 |
| V (B3) [mL] | 0 | 0.5 | 1.0 | 2.5 | 5.0 |
| V (DMF) [mL] | 5.0 | 4.5 | 4.0 | 2.5 | 0 |

TABLE 6
Production of the Energy Transfer Solutions C4a-C4e

| Solution | C4a | C4b | C4c | C4d | C4e |
|---|---|---|---|---|---|
| V (A) [mL] | 5.0 | 5.0 | 5.0 | 5.0 | 5.0 |
| V (B4) [mL] | 0 | 0.5 | 1.0 | 2.5 | 5.0 |
| V (DMF) [mL] | 5.0 | 4.5 | 4.0 | 2.5 | 0 |

2. Production of Phosphorescent Nanoparticles

For producing the particles, 1 g of the polyacrylonitrile/polyacrylic acid-copolymer is dissolved in 200 ml dry diemthylformamide. 20 mg of the donor dyestuff with varying percentages of the respective acceptor dyestuff were dissolved therein. Thus, 400 ml distilled water were added by dripping, in order to precipitate the polyacrylonitrile (PAN) as nanoparticles. A clear phosphorescent solution is thereby obtained. After 1 hour of waiting, a normal HCl solution is added, in order to allow the dyed particles to aggregate. Thereafter, the so obtained suspension is spun and washed with distilled water. The precipitate is suspended in a phosphate buffer of pH 7.0 and redispersed under ultrasound. After warming to 70° C. for 15 minutes, the suspensions were clear and remained stable over several weeks. They were stored, protected from light, at 10C.

2. Measurement Design

A donor dyestuff and several acceptor dyestuffs are embedded within the same polyacrylonitrile-nanoparticle, so that an energy transfer between the dyestuffs can be realized. The additional reactive carboxyl groups at the surface of the particle simplify the coupling of the nanoparticles via covalent bonds to proteins and other biomolecules.

Corrected fluorescence-emission spectra for computing the quantum yield were obtained by using the following equation (1). Hereby, Φ is the quantum yield, A (λ) is the absorption per centimeter of solution at the excitation wavelength λ, I(λ) is the relative intensity of the excitation light at the wave length λ, n is the average computation index of the solution for the luminescence and D is the surface integral under the corrected emission spectrum. The indexes x and R refer to the unknown respectively the reference (ruthenium(II)tris(2,2-bipyridyl) chloridehexahydrate)-solutions.

(Formula p.12)

Since during the measurements of the quantum yield, the voltage of the detector was kept constant, and since all solutions were watery solutions, the following simplifications could be carried out:

I(λ_{R})≈I(λₓ) and nₓ≧n_{R}.

Multiple frequency-phase measurements (1 kHz to MHz) were carried out with an ISS K2-multiple frequency-phase fluorometer. The decay period measurements were done in the frequency domain. Average decay periods τ were computed from the phase angles θ, which were obtained through single frequency measurement, in accordance to the following equation (2)

$$τ = \frac{tan θ}{2 π f}$$

For light source a bright blue light-emitting diode (LED) (λₘₐₓ=470 nm, NSPB 500, Nichia Nürnberg, Germany) was used, outfitted with a blue glass filter (BG 12, Schott, Mainz, Germany). As detection unit, a compact red-sensitive photomultiplier tube was used (H5701-02, Hamamatsu, Herrsching, Germany), outfitted with a rejection filter (OG 570, Schott). The excitation light of the LED was sinus wave-modulated at a frequency f of 45 kHz by using a double phase lock-in-amplifier (DSP 830, Stanford Research, Sunnyvale, Calif., USA).

The amplifier was also used for measuring the phase shift of the emitted luminescence. A forked fiber bundle with glass fibers (NA 0.46, d=2 mm) was coupled to a thermostatic cell (T=25° C.), wherein the tip of the fiber bundle was dipped into the agitated measuring solution.

4. Choice of the Matrix and the Dyestuffs

The poly(acrylonitril-co-acrylic acid)copolymer is an excellent matrix, since it has a low gas permeability and thus protects the embedded luminescence dyestuffs from gas, such as oxygen, which leads to negligible quenching effects. Furthermore, the carboxyl groups provide the copolymer with reactive groups for covalent bonding to other molecules.

Polyacrylonitrile-derivatives form a suitable matrix for embedding organic phosphorescent dyestuffs, since they have a small permeability for gases and dissolved ionic and neutral chemical compounds. Thus, the dyestuffs are efficiently protected against luminescence quenching, for example due to molecular oxygen, and thus exhibit constant decaying periods and quantum yields in samples of variable and unknown compositions. Additionally, many lipophilic dyestuffs are well soluble in these materials and are not washed out into the sample.

The nanoparticles have a very high surface volume ratio. Polyacrylonitrile with a polyacrylic acid content of 5% has shown to be an especially useful embedding matrix. Suspensions of such phosphorescent nanoparticles are practically not quenchable through oxygen, they exhibit no sedimentation tendency and have an activated surface for coupling of biomolecules or chemically reactive indicators. In case of using the ruthenium-(II)-tris(4,7-diphenyl-1,10-phenantroline)-complex as a phosphorescent dyestuff, bright luminescent nanoparticles are obtained having strong Stokes-shifts. In watery solutions no washout of dyestuffs could be observed. They can either be excited by a blue argonionlaser or with bright bluelight-emitting diodes (LED's).

The precipitation process affords the simultaneous embedding of various phosphorescent and fluorescent dyestuffs in an individual nanoparticle.

The long-living phosphorescent luminescence donor Ru(dph-phen)₃ (TMS)₂ exhibits a great Stokes-shift of about 150 nm (λₓ=467 nm, λₘ=613 nm), a high quantum yield (φ>40%), a large extinction coefficient (ε=28, 100 LMol⁻¹×cm⁻¹), and is lipophilic, in order to avoid a dilution in watery surrounding. It can be excited by means of an argon-ion laser at λₓ=488 nm. Finally, its emission spectrum is broad enough to overlap with the absorption spectra of various luminescent acceptor dyestuffs. During the production process, they are completely incorporated into the particle.

Fluorescent carbocyanines act as luminescence-energy acceptors. The advantage of these indicator dyestuffs, is that they show no inherent absorption at the excitation wavelength of the ruthenium complex of 488 nm. Due to their high extinction coefficient ε of more than 200,000 LMol⁻¹ cm⁻¹, their lipophilic character, their great overlapping integrals with the ruthenium donor-dyestuff and finally their easy commercial availability, render the carbocyanine dyestuffs as ideal energy acceptors.

Table 7 summarizes the spectral data of the donor-and acceptor dyestuffs in DMF used here.

TABLE 7
Spectral Characterization of the Ruthenium Donor and Carbocyanine
Acceptor Dyestuffs.

| Dyestuff | Solvent | λₘₐₓ(nm) | λₑₘ(nm) | Δλ(nm) | ε(L moL⁻¹ cm⁻¹) |
|---|---|---|---|---|---|
| RU^{a} | phosphate | 465 | 612 | 147 | 28.100 |
| | buffer | | | | |
| CY582 | DMF | 587 | 608 | 21 | 224.700 |
| CY604 | DMF | 612 | 633 | 21 | 238.300 |
| CY655 | DMF | 659 | 678 | 19 | 245.400 |
| CY703 | DMF | 713 | 731 | 18 | 324.500 |

^{a}enclosed in nanoparticles (= solution C1a)

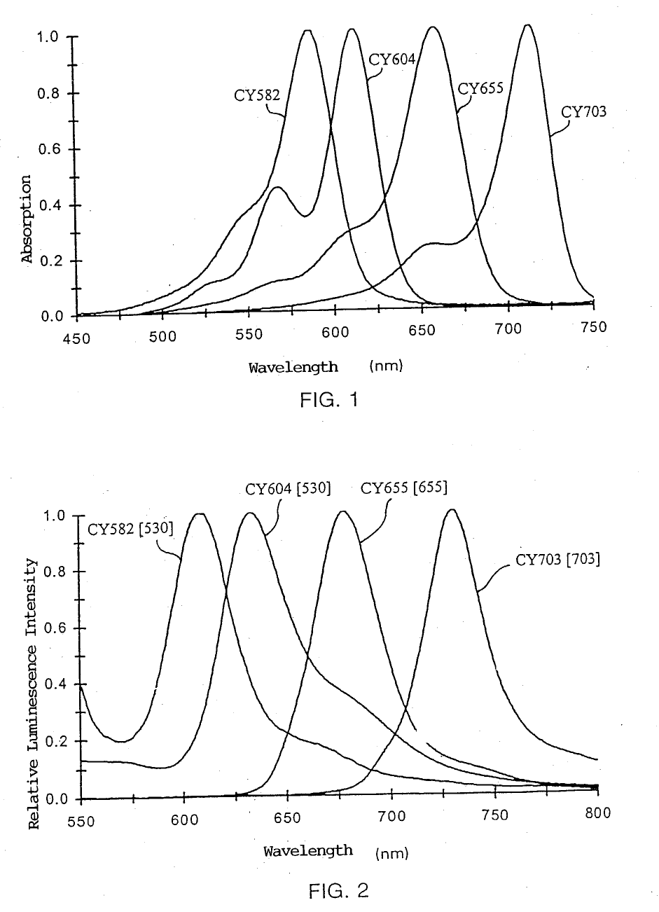

The FIGS. 1 and 2 show normalized absorptions-and fluorescence emission spectra of the carbocyanine dyestuffs utilized in DMF.

A two-dimensional arrangement of multiplex markers is obtained, wherein the first dimension is the absorption wavelength λ of the carbocyanine-acceptor dyestuffs and the second dimension is the luminescence-decay period τ.

Seven or eight different carbocyanine dyestuffs can even be utilized as luminescence energy-acceptors, as long as their excitation wavelength covers the ruthenium-donor emission wavelength in the range of approximately 590 nm to 750 nm. Through spectral overlap of a dyestuff pair, energy transfer is possible and phosphorescence is transferred to fluorescence indicators, such as the longwave excitable carbocyanine dyestuffs. Thus, phosphorescent nanoparticles with an exceptionally large Stokes-shift up to 300 nm can be produced. These nanoparticles can be utilized as bright phosphorescent markers in the immuno-or DNA-sensitizing or as nanoprobes for measuring intracellular chemical parameters. Furthermore, they form excellent phosphorescence standards and are useful for the design of phosphorescent chemical sensors.

5. Characterization of the Nanoparticles

Table 8 shows a summary of the spectral characterization of four different carbocyanine-nanoparticles with varying dyestuff concentrations in phosphate buffer solution (pH 7.0; IS=20 mmol).

TABLE 8
Nanoparticles-Characterization of the Ruthenium Donor-Carbocyanine
Acceptor Pairs in Phosphate Buffer Solution

| | Carbocyanin | c (acceptor)^{a} | τ, air | | | Δφ |
|---|---|---|---|---|---|---|
| Solution | Acceptor | (μmol/L) | (μs) | φ, air | φ, Na₂SO₃ | (%) |
| C1a | CY582 | 0 | 6.23 | 0.37 | 0.39 | −5.1 |
| C1b | CY582 | 4.11 | 5.14 | 0.36 | 0.37 | −2.7 |
| C1c | CY582 | 8.22 | 4.58 | 0.34 | 0.35 | −2.9 |
| C1d | CY582 | 20.56 | 2.59 | 0.32 | 0.33 | −3.0 |
| C1e | CY582 | 41.12 | 1.03 | 0.27 | 0.28 | −3.6 |
| C2b | CY604 | 5.14 | 2.87 | 0.32 | 0.32 | > −1.0 |
| C2c | CY604 | 10.28 | 1.77 | 0.25 | 0.25 | > −1.0 |
| C2d | CY604 | 25.71 | 0.63 | 0.08 | 0.08 | > −1.0 |
| C2e | CY604 | 51.42 | 0.39 | 0.03 | 0.03 | > −1.0 |
| C3a | CY655 | 0 | 6.00 | 0.32 | 0.33 | −3.0 |
| C3b | CY655 | 3.86 | 4.15 | 0.30 | 0.31 | −3.2 |
| C3c | CY655 | 7.71 | 1.78 | 0.27 | 0.28 | −3.6 |
| C3d | CY655 | 19.29 | 0.85 | 0.18 | 0.19 | −5.3 |
| C3e | CY655 | 38.57 | 0.38 | 0.07 | 0.08 | −12.5 |
| C4b | CY703 | 11.10 | 3.97 | 0.25 | 0.26 | −3.8 |
| C4c | CY703 | 22.20 | 2.56 | 0.20 | 0.21 | −4.8 |
| C4d | CY703 | 55.51 | 1.46 | 0.16 | 0.16 | > −1.0 |
| C4e | CY703 | 111.02 | 1.16 | 0.06 | 0.06 | > −1.0 |

^{a}c(RU-donor) 10.00 μmol/L (constant)

Here, in the third column c means the concentration of the acceptor, τ in the fourth column, the decay period, and φ in the fifth and seventh column, the quantum yield.

The resulting two-dimensional field of multiplex-markers shows similar features when excited with an argon ionlaser at 488 nm. The average decay period increases in dependence of the carbocyanine and its concentration utilized.

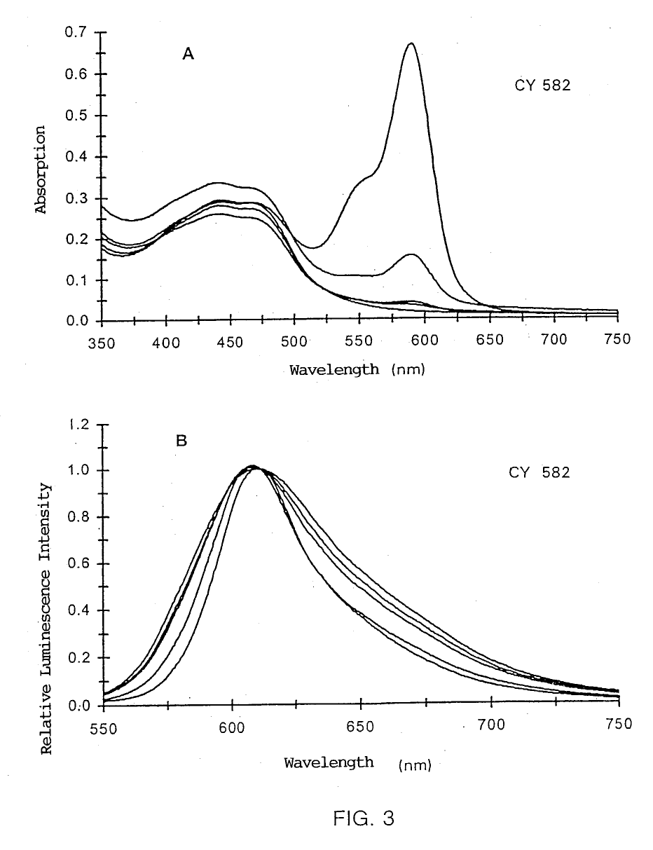

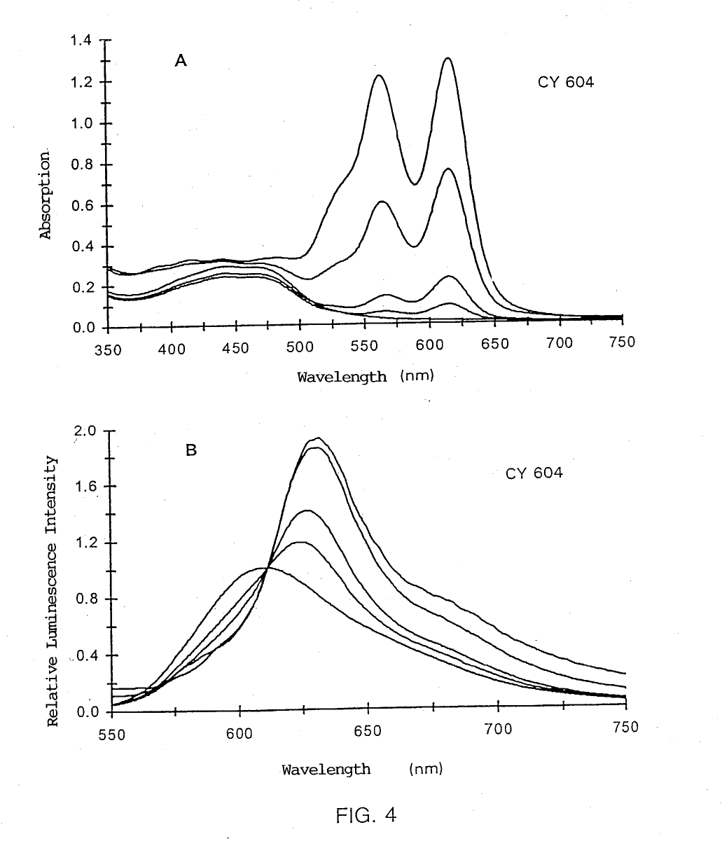

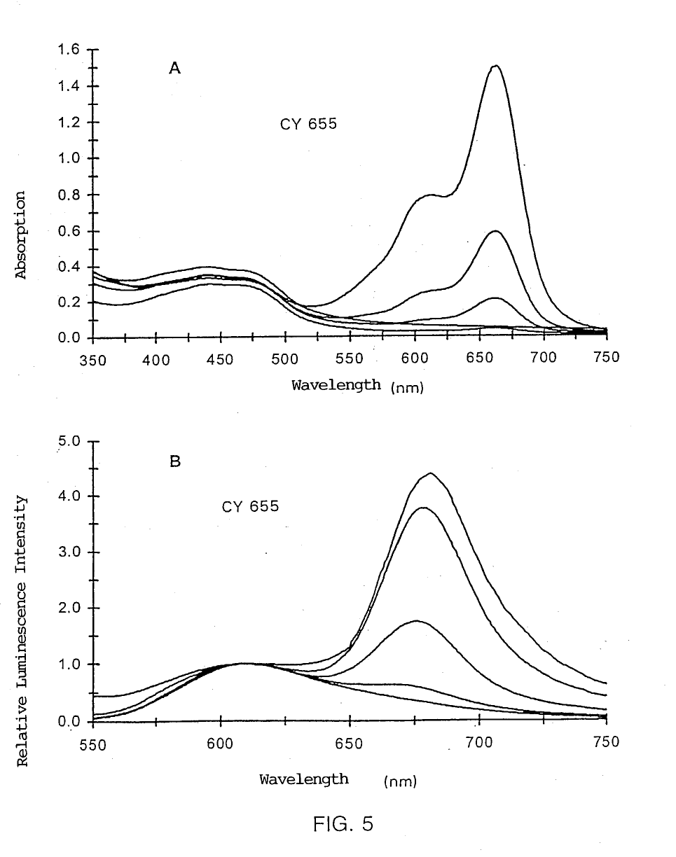

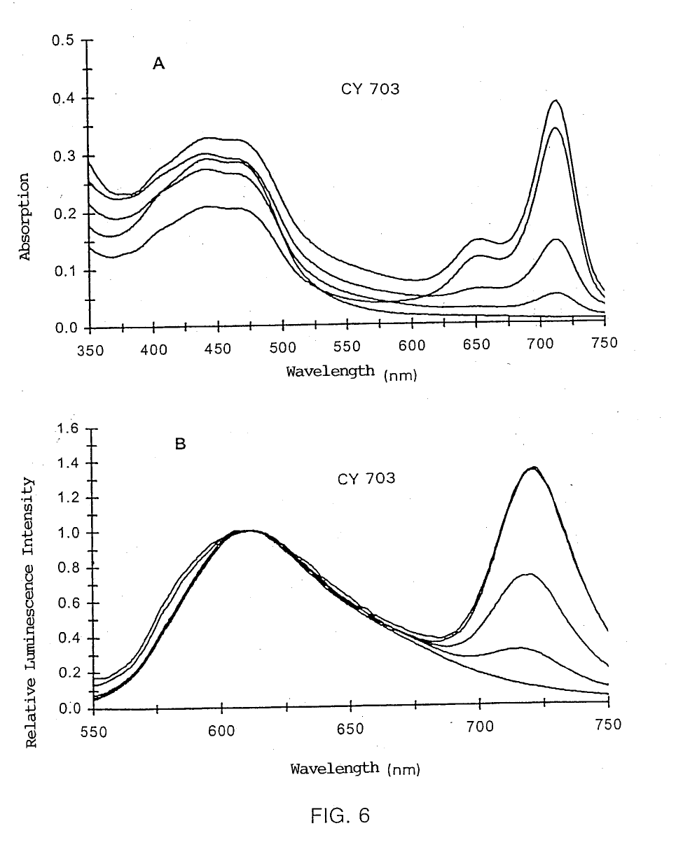

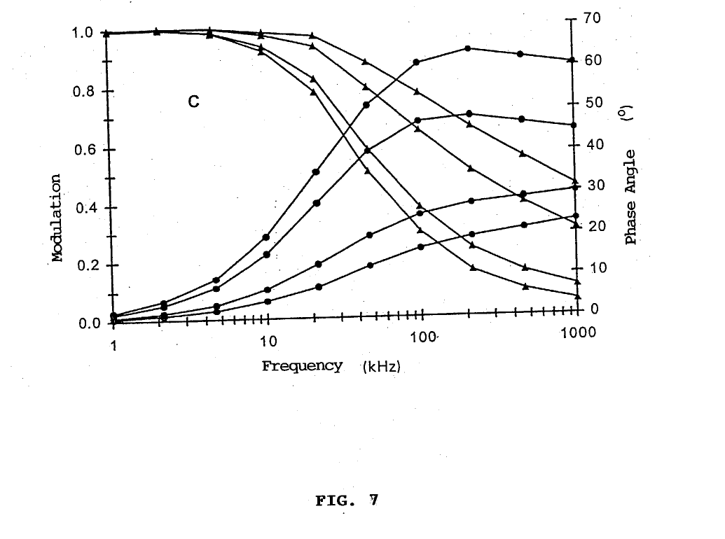

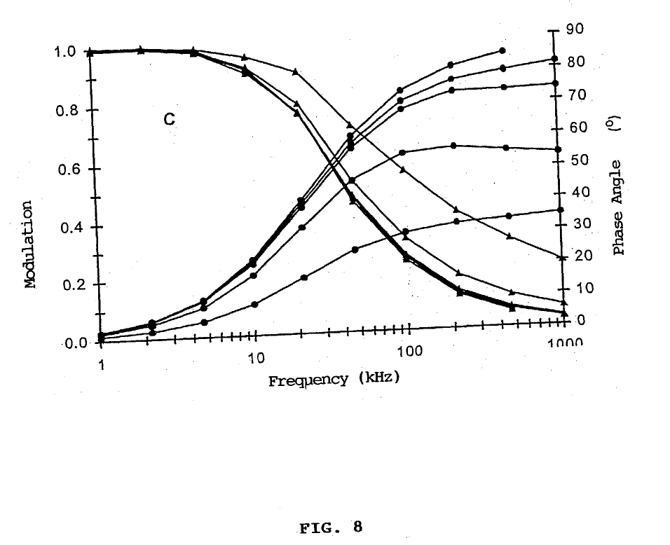

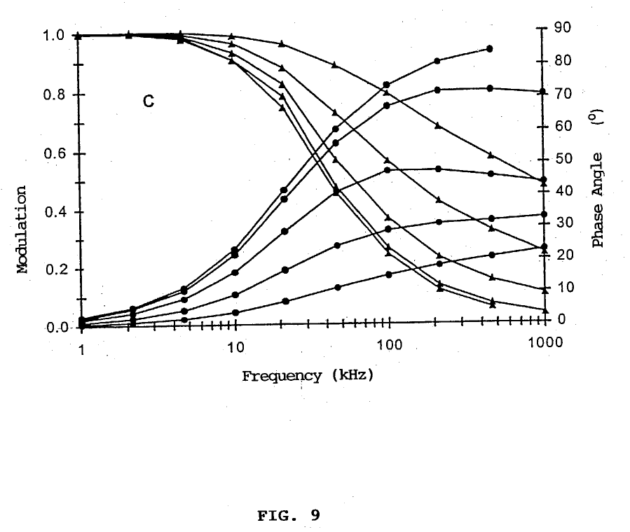

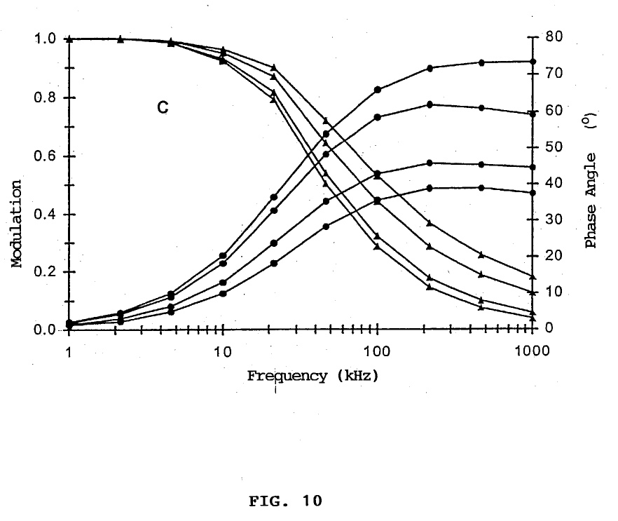

The FIGS. 3 to 6 each show above (A) the absorption spectra of the nanoparticles for each type of different carbocyanine (CY562, CY604, CY655 and CY703), each with different concentrations in phosphate buffer solution, and each below (B) the emission spectra (λₓ=488 nm) of each particle, which are being normalized to 1 at the emission wavelength of the ruthenium donor complex (611.5 nm). The FIGS. 7 to 10 each show the phase angle and the modulation in a frequency range of 1 kHz to 1 MHz of the particles in FIGS. 3 to 6.

The fluorescent emission of the ruthenium donor complex decreases due to the energy transfer to the carbocyanine acceptor in one and the same nanoparticle. Furthermore the photo-physical properties were examined, namely the tendency of the nanoparticles to aggregate and their stability. In phosphate buffer solution at pH 7.00 with an ionic strength (adjusted with NaCl of 20 mmol), the particles were stable over the course of several weeks. The suspensions should be stored protected from light and at about 10° C.

In addition to the spectral characterizations of the particles, their physical properties were examined. Grid-electronmicroscopic pictures of the particles show an almost circular shaped form and a diameter of about 50 nm. The static and dynamic light scattering at laser Doppler-anemometric-experiments resulted in a polydispersed coil with a particle diameter from 100 to 50 nm and a zeta-potential, which confirmed the negative surface charge due to carboxyl-groups, as shown in Table 9.

TABLE 9
Particle Size and Surface Charge of the Solution C3a at Dynamic
Light Scattering Experiments.

| c(Ru(dph- | | | |
|---|---|---|---|
| phen)₃(TMS)₂) | c(CY655) | hydrodynamic diameter | ζ-potential |
| [μmol] | [μmol/l] | [nm ] | [mv] |
| 39.6 | 0.0 | 84.7 | −58.0 ± 0.7 |

1. Arrangement for fluorometric measurement of an analyte comprising:
a luminescent donor dyestuff and several acceptor dyestuffs that are immobilized together with the donor dyestuff and that luminesce through energy transfer from the donor dyestuff,
characterized in that the donor dyestuff is a phosphorescence dyestuff and the respective acceptor dyestuffs are fluorescence dyestuffs.

2. Arrangement according to claim 1, characterized in that only one single donor dyestuff is immobilized.

3. Arrangement according to claim 1 or 2, characterized in that the donor dyestuff is reacting to blue light and preferably is a ruthenium-(II)-polypyrid complex.

4. Arrangement according to one of the preceding claims, characterized in that the several acceptor dyestuffs exhibit distinctive emission spectra or/and in interaction with the donor dyestuff induce distinctive luminescence decay periods of the arrangement.

5. Arrangement according to one of the preceding claims, characterized in that an acceptor dyestuff is provided separately each in varying concentrations immobilized with the donor dyestuff.

6. Arrangement according to one of the preceding claims, characterized in that the acceptor dyestuffs each are carbocyanine dyestuffs.

7. Arrangement according to one of the preceding claims, characterized in that into a plastic matrix that is a donor dyestuff- and the acceptor dyestuff-immobilizing matrix, the donor dyestuff is embedded with a concentration of 1 to 15% by weight, preferably about 10% by weight.

8. Arrangement according one of the preceding claims, characterized in that the donor dyestuff and the acceptor dyestuffs are embedded into micro- or nanoparticles, preferably in a size in the range of 50 μm.

9. Arrangement according to claim 8, characterized in that the micro-or nanoparticles are produced by precipitating a solution of polynitril in dimethylformamide (DMF).

10. Arrangement according to one of claims 1 to 9, characterized in that the donor dyestuff exhibits a luminescence decay period in the range of 100 ns to 100 μs, preferably 100 ns to 10 μs.

11. Arrangement according to one of claims 1 to 10, characterized in that the acceptor dyestuffs exhibit a luminescence decay period of ≧50 ns, preferably 50 ns to 10 μs relative to the luminescence stimulated by the donor dyestuff.

12. Method for simultaneous fluorometric measurement of several analytes, in particular by means of an arrangement according to one of the preceding claims, which exhibits a phosphorescent donor dyestuff and several distinct fluorescent acceptor dyestuffs immobilized herewith, with the steps of:
exciting the donor dyestuff,
measuring and evaluating the spectrally differing fluorescence responses of the acceptor dyestuffs and
measuring and evaluating the luminescence decay periods, which are influenced by the fluorescence signals of the acceptor dyestuffs in interaction with the donor dyestuff.

13. Method according to claim 12, characterized in that a time-resolved measurement and evaluation of the fluorescence responses of the acceptor dyestuffs or/and the luminescence decay periods occurs, in order to reduce the background signals.
